(19) United States
(12) Patent Application Publication
(10) Pub. No.: US 2002/0133215 A1
(43) Pub. Date: Sep. 19, 2002

(54) Profreez sportswrap

(76) Inventor: Phillip W. Kubec, Parkland, FL (US)

Correspondence Address: HENRY NOVAK, 6306 NW 72nd Place, Parkland, FL, 33067, US

(21) Appl. No.: 09/809,149

(22) Filed: Mar. 16, 2001

(51) Int. Cl. A61F 7/00

(52) U.S. Cl. 607/109

(57) ABSTRACT

Fastening neckband with instant cool or heat transfer device, cloth-like re-usable wrap that has a pocket lengthwise centrally located for receiving an elongated cool or heat transfer element in the form of an instant, reusable coolant or heating transfer device therein. The lengths of the main body pocket are chosen to place the reusable cooling or heat transfer element about the back and sides of the neck, while leaving the main body unobstructed at free ends to permit loose securing of the ends in front of the neck via a reusable Velcro® fastener.

### BACKGROUND OF THE INVENTION

It is standard to employ heat transfer material in packs to be wrapped about body parts for cooling or heating purposes. The application of hot or cold packs about the neck and face has proved effective for alleviating sore muscles, headaches, fever and similar ailments; and, in general, for increasing the overall well-being and comfort of the user. Such packs may employ water or another reusable cooling or heating medium, or may utilize a compartmentalized formulation to produce an endothermic or exothermic reaction.

A typical conventional heat transfer device of the type to which the invention relates comprises of a hollow tubular neckband of single layer flexible fabric material or plastic which is wrapped about the releasable fastener. A heat transfer element, such as a flexible liquid-tight pouch containing reusable refrigerant, is inserted in the hollow of the neckband for heat exchange with the neck through the layer of neckband material.

Such tubular neckband wraps are clumsy in appearance, giving a dog collar look that is inappropriate for social wear. The releasable fastener is uncomfortable and awkward to adjust; and no mechanism is provided for varying the thickness of neckband material lying between the heat transfer element and the user's skin.

### BRIEF SUMMARY OF THE INVENTION

Fastening neckband with an insert of cool or heat transfer device, cloth-like re-usable wrap which has a pocket lengthwise centrally located for receiving an elongated, flexible, instant cool or instant heat transfer element in the form of a gellpack reusable coolant or heating pouch therein. The lengths of the main body pocket are chosen to place the reusable cooling or heat transfer element about the back and sides of the neck, while leaving the main body unobstructed at free ends to permit loose securing of the ends in front of the neck via a reusable Velcro® fastener.

### DETAILED DESCRIPTION OF THE INVENTION

The present invention provides for an instant, heat or cold transfer device in the form of a stylish band-like sportswrap, having free ends, which can be releasable, joined together by a Velcro® fastener and having an aesthetically pleasing look. The invention further comprises a neckband heat or cold transfer device adapted to permit variance, as desired by the user, of the number of layers of material between the heat or cold transfer element and the skin.

The heat or cold transfer device of the invention comprises a main body of flexible fabric material having a length and width, and a pocket lengthwise centrally located relative to the main body and into which a heat transfer element can be placed. The pocket is dimensioned, configured and adapted so that the received instant cooling or instant heat transfer element placed in the pocket will extend sufficiently lengthwise of the device to envelop the back and sides of the neck, but leave sufficiently unobstructed opposite free ends lengthwise of the main body, to permit securing the main body at those free ends in front of the neck. The pouch holding the heat or cold transfer device is designed to allow the pouches to be inserted into each other so that single or multiple layers of fabric material may be optionally located intermediate the heat or cool element and the skin.

The elongated rectangular pocket having an opening with a releasable closure is located lengthwise centrally of a main body to extend for less than a major portion of the main body, both lengthwise and widthwise thereof.

The heat phase of the heat transfer device consists of a chemically generated heating element whereby the heat is released instantly upon the breaking of the element within the plastic liquid-tight protective cover. Thereafter, the element is inserted into the wrap. It provides heat to the affected area for a period of up to two hours. The heat transfer device is reusable.

The cooling phase of the transfer device consists of a gel-like substance which is placed in a plastic liquid-tight protective plastic. In its initial usage, the cooling phase begins instantly upon the breaking of the element in the liquid-tight protective cover. Prior to its repeated usage, the flexible, gel-like pack is frozen in a regular household freezer for a period of two hours.

The invention varies from what is currently available on the market because it is instant, reusable and flexible so that it may conform to the user's needs.

1. A transfer device of cooling or heating the neck area of a user, said device comprising:

2. A main body of flexible fabric material having a length and width;

3. A pocket of flexible fabric material, having a length, width and an interior, and being located lengthwise centrally of the length of said body; and

4. An instant and reusable heat or cold transfer element dimensioned, configured and adapted to match said pocket interior and including substance suitable for transferring heat or cooling to or from said neck area;

5. Said lengths of said main body and pocket being relatively dimensioned for enabling said pocket-matching heat or cooling transfer element to be received circumferentially about the back and side of the neck, yet leaving opposite free ends lengthwise of said main body and away from said pocket, to permit securing via a Velcro® attachment in front of said neck; and

6. Said body and pocket being relatively dimensioned for permitting said items to be inserted into each other to permit optional user selectable variation of the number of layers of fabric material between said heat or cool transfer element and said neck area.
